(19) United States
(12) Patent Application Publication
(10) Pub. No.: US 2019/0328718 A1
(43) Pub. Date: Oct. 31, 2019

(54) Medicaments and Uses in Treatment of Cancer and other Pathological Conditions Associated with Aging

(71) Applicant: Sinan Tas, Bor (TR)

(72) Inventor: Sinan Tas, Bor (TR)

(21) Appl. No.: 16/501,847

(22) PCT Filed: Mar. 24, 2017
(86) PCT No.: PCT/TR2017/000043

§ 371 Date: Jun. 17, 2019

Related U.S. Application Data
(63) Continuation-in-part of application No. 15/530,513, filed on Jan. 23, 2017, now abandoned.

(51) Int. Cl. A61K 31/4355; A61K 9/00

(52) U.S. Cl. CPC A61K 31/4355; A61K 9/0019

(57) ABSTRACT

This invention concerns rational therapeutic interventions with age associated pathological conditions. In one aspect a drug treatment of tumor bearing human is described that provides elimination of tumor cells from patients independent of the histopathological class and stage of tumor.

### BACKGROUND OF THE INVENTION

Cancer is a disease associated with aging. Records in human populations around the world show increased occurrence of cancer in older age groups. Longitudinal studies of cohorts also show an age associated increase in the incidence of tumors originating from various organs and cell types. Investigations with experimental animals confirm the findings in human that tumorigenesis is commonly associated with aging and show that it is a multistep process requiring occurrences of multiple genetic and epigenetic changes in cells. Exposures to particular environmental agents through diet and other routes are found to increase the probability of tumorigenesis at younger ages. Genotype of an individual can affect his or her susceptibility to tumor development significantly.

Patients diagnosed to have cancer are usually treated with surgical excision of tumor when it is feasible. When surgery is considered not likely to save the life of patient, e.g. due to the location or stage of tumor, or when it is not desirable (e.g. a mutilating surgery), various drug and/or radiation treatments are used and when they are considered unlikely to help the patient, the patient is comforted by pain-killing and other support. Radiation treatments generally involve delivery of radiation to the anatomical location having the neoplastic mass and cause radiation damage to the genetic material of cells in the field of radiation. When such damage is extensive tumor cells can be killed by apoptosis (a programmed cell death process) and other effects may also occur depending on radiation dose. Normal cells of the patient can also be harmed and killed by radiation and it represents a shortcoming of this approach to treatment of cancer patients. Clinical experience shows that a minority of all tumor bearing patients can be cured by radiation treatments, the treatment results are largely dependent on the organ of origin, histopathological class and stage of tumors and many of them are resistant to radiation treatments. Most of the conventional drug treatments of tumor bearing patients are known to act similar to the radiation treatments of cancer, by causation of damage to the genetic material of cells although other modes of effect have also been determined for different drug treatments. For example causations of reduced proliferation of tumor cells or necrosis have been described following certain drug administrations. Necrotic cell death is distinct from apoptosis and causes release of most intracellular contents including the macromolecules to the tissue environment due to damaged plasma membrane and triggers inflammatory reactions. Effects of radiation or drug administration on the normal cells and functions of a tumor bearing patient are in general critical with regard to the therapeutic outcome. For example it is clear from investigations with experimental animals and from observations in human exposed to excessive radiation due to accidents that harming of the normal cells and lowering of normal functions below a threshold even in a single organ can be fatal and the risk of death increases when multiple organs are affected to preclude a beneficial therapeutic result.

A further dilemma with the radiation and drug treatments of cancer that act by causation of extensive damage to the genetic material of tumor cells is that even when cure is achieved as it has been observed in a minority of all surgically incurable patients, the probability of occurrences of new cancers is increased many folds in these patients during their follow up in comparison to age-matched control populations and it is due to the genetic and epigenetic changes caused in the normal cells of patients by such treatments.

Cancer is a leading cause of death not only in human but also in other investigated species, in individuals living without predation to an age approaching to the species-specific maximum lifespan potential. Maximum lifespan potential is a genetically affected trait. It is shorter in species having faster aging. For example mice show a far greater rate of aging than human and mice in their third year of life are close to the end of their natural lifespan and commonly die with a tumoral disease.

Aging is found to be associated with increasing probabilities of occurrences of various other diseases besides cancer as described in medical textbooks. Cancer and they collectively constitute major causes of decline of working capacity and health in human. A basic principle in medicine is that effective treatment of a particular disease requires a working solution of the pathogenesis of that disease at molecular, cell-tissue and whole organism levels. Tumorigenesis is found to be just one of the pathologies associated with biological aging and yet existing technologies in dealing with them are inadequate as shown by clinical results, epidemiological data and health records published by governments. The answer to the question whether or not there are living organisms that do not show signs of aging is affirmative. Biological aging is a feature of eukaryotic organisms; even the unicellular eukaryotic organisms show signs of clonal aging. On the other hand these free living eukaryotic cells and the germ lines of multicellular eukaryotes, including human, are capable of minimizing aging damages to the genetic material and effectively repair such damage during meiosis and the prokaryotic organisms do not show aging (Taş S, Monographs In Developmental Biology 1984; 17:178-192). Prokaryotes and eukaryotes, while showing identities and major similarities of their molecular constituents and molecular reactions, differ significantly in the structure of their genetic material. DNA in eukaryotes is linear and extensively complexed with particular proteins and other macromolecules to make the chromatin complex.

Cancer cells show the remarkable feature of avoiding senescence under conditions where normal cells show signs of aging. Neoplastically transformed cell populations can be propagated indefinitely in vitro and also in syngeneic hosts in vivo. Tumor cells can be serially passaged without limit in inbred animals that do not show histoincompatibility with the transplanted tumor cells. Normal cells show limited lifespan under the same conditions in vitro and in vivo. Chromatin is a giant macromolecular complex with multiple components. Certain of its constituents interact with a nuclear skeletal structure variously called as nuclear matrix-lamina that in turn interact with the nuclear membrane, the nuclear pore complex and cytoskeleton. Comparisons of the structure of chromatin complex in normal cells and cancer cells have revealed consistent differences between them that accord with the escape of cancer cells from senescence (Taş S, Monographs In Developmental Biology 1984; 17:178-192).

Multiple lines of evidence show that prokaryotes originated earlier than eukaryotes in a world with little oxygen in its atmosphere and horizontal gene transfer and symbiosis events among the early bacteria led to the eukaryotes. The earliest mitochondrion-containing eukaryote appears to have occurred from a symbiosis event at a time when earth's atmosphere had become oxidizing similar to today's atmosphere Hedges S B et al, BMC Evolutionary Biology 2001; 1:4). The oxidative energy metabolism enabled by mitochondria provides far greater numbers of ATP from glucose than that occurs without oxidative metabolism but the generation of oxygen radicals and other oxidizing species in mitochondria is also a threat to the maintenance of the integrity of genome that must be dealt in eukaryotes.

Sequencing of the human genome has unambiguously demonstrated a further threat to the maintenance of the integrity of genome in human and other eukaryotes. Majority of the nucleotide sequences in the human genome apparently originated from anciently introduced sequences that are repeated throughout the genome. These transposon and retrotransposon derived repeated sequences can be seen as consequences of ancient parasitic events as it is clear that there have been measures to prevent their spreading in the genome and that most but not all have been inactivated in the human genome (Lander E S et al, Nature 2001; 409:860-921). The faster aging mouse is found to be less successful than human in dealing with these repeated sequences and in preventing their spreading in the genome (Lander et al, ibid).

Heterochromatinization of the genomic regions containing the transposon-retrotransposon sequences and other repeated sequences is a strategy developed against them. Such heterochromatinic regions are commonly called as constitutive heterochromatin. Facultative heterochromatinization refers to the heterochromatinization of the particular gene domains during cellular differentiation. It provides silencing of the affected genes. Inactivation of one of the two X chromatins in female mammalian embryos early during development during the differentiation from embryonal stem cells is a known example of such facultative heterochromatinization. Constitutive heterochromatinization and facultative heterochromatinization show numerous of their molecular components and mechanisms of heterochromatinization shared. Replication of repeated sequences is known to carry higher risks of replication errors than of non-repeated sequences and the need to silence the transposon-retrotransposon derived repeated sequences poses additional challenges in terms of the repair of damage to the genetic material at heterochromatinic regions. Facultative heterochromatinization is required for cellular differentiation but there is evidence that both facultative heterochromatin and constitutive heterochromatin structure cause restrictions to the repair of damage to the genetic material (Taş S, Gerontology 1978; 24:358-364; Taş S, Monographs In Developmental Biology 1984; 17:178-192). The increase of mutations from unrepaired and misrepaired damages to the genetic material during aging and their occurrences in much greater frequencies in heterochromatin than in euchromatin, revealed e.g. by comparisons of such between the two X chromatins in the same cell in aging women (e.g. Abruzzo M A et al, Cytogenetics and Cell Genetics 1985; 39:275-278; Machiela M J et al, Nature Communications 2016; 7:11843), is testimony to that restriction and challenge.

The avoidance of senescence by most cancer cells in vitro and in vivo is associated with distinct chromatin structure changes in them (Taş S, Monographs In Developmental Biology 1984; 17:178-192; Taş S et al, Cytologia 1985; 50:405-415). But, unlike the elaborate measures for maintaining the genome's integrity in germ line cells, the neoplastic cells escaping senescence are not found to be maintaining genome's integrity. To the contrary neoplastic cells show in general genetic instability.

The hedgehog gene was found first in a systematic screen of the genes that affect pattern formation during embryonal development (Nüsslein-Volhard C et al, Nature 1980; 287:795-801). It has subsequently been determined to be largely conserved in species from drosophila to human and to encode for a secreted processed polypeptide (abbreviated here as Hh) that induces differentiation of particular cell types during development and also during postnatal life. Binding of Hh to a transmembrane protein, Patched, on a receiving cell initiates a molecular signaling transduced in the cell by another transmembrane protein, Smoothened (abbreviated here as Smo). When not liganded by Hh, Patched inhibits the signaling activity of Smo and the binding of Hh to Patched relieves the inhibition of Smo by Patched. Signaling activity of Smo can be stimulated also in a Hh ligand-independent manner, e.g. by mutational events that cause loss of function of Patched. The signaling by the relieved Smo acts to regulate transcriptions of Hh target genes through actions of Ci/Gli transcription factors (Ci in drosophila and its homologs Gli1, Gli2, Gli3 in vertebrates). The Smoothened protein has been determined to be essential for the signaling initiated by Hh in diverse species (e.g. Struhl G et al, Development 1997; 124:2155-2165; Zhang X M et al, Cell 2001; 105:781-792). In response to Hh/Smo signaling the Ci/Gli proteins are translocated to the cell nucleus where they execute transcriptional effects of Hh/Smo signaling.

Several protein kinases have been identified that phosphorylate particular aminoacids of Ci/Gli proteins and affect their proteolytic processing and functioning. These include protein kinase A (PKA), glycogen synthase kinase 3 (GSK3), casein kinase 1 and homeodomain-interacting protein kinase (Pan Y et al, Mol Cell Biol 2006; 26:3365-3377; Swarup S et al, Proc Natl Acad Sci USA 2011; 108:9887-9892). Stimulation of PKA activity by increase of intracellular cAMP has been found to promote proteolysis of Ci/Gli proteins and to be inhibitory on expressions of Hh target genes. PKA is not however suitable for selective inhibition of Hh/Smo signaling at least because of the various other substrates of PKA that are unrelated to Hh/Smo signaling. Experimental results show in this respect that effects of PKA are not the same as that caused by a selective genetic elimination of Smo even in the same cells of an animal (Wang Q T et al, Development 2000; 127:3131-3139). Protein kinases having substrates besides those involved in Hh/Smo signaling do not serve for a selective action on Hh/Smo signaling. For example GSK3 is found to phosphorylate β-catenin to affect Wnt/β-catenin signaling and itself is target of ras and phosphatidyl inositol 3 kinase-AKT pathways (e.g. Diehl J A et al, Genes & Development 1998; 12:3499-3511). Hence a molecular signalling other than Hh/Smo can affect amount and functioning of Ci/Gli proteins in cells through e.g. above mentioned protein kinases but it does not equal to what is performed by Hh/Smo signaling. The signaling initiated by Hh in a receiving cell is on the other hand transduced necessarily by Smo and the transcriptional effects of Hh/Smo signaling occur obligatorily through Ci/Gli transcription factors (e.g. Methot N et al, Development 2001; 128:733-742). These features make Hh/Smo signaling well suited to selective inhibition.

Smo, besides signaling to affect transcriptions of Hh target genes, exerts also rapid effects that are not mediated by Ci/Gli. For example a guanine nucleotide exchange factor, Tiam1 (T lymphoma invasion and metastasis 1) protein, is found to interact with the C terminal region of Smo and dissociates from Smo in response to Hh arrival to the cell and causes cytoskeletal remodeling and membrane ruffles (Sasaki N et al, Molecular and Cellular Neuroscience 2010; 45:335-344). Likewise interaction of Smo with GTP-binding protein α-1 (Gαi), their co-localization in the primary cilium in neurons responding to Hh and rapid Hh-induced Ca⁺⁺ spiking activity via Smo and Gαi have been described to occur independent of a transcriptional effect of Hh/Smo signaling (Belgacem Y R et al, Proc Natl Acad Sci USA 2011; 108:4482-4487). The described rapidity of the Hh/Smo signaling-induced Ca⁺⁺ spikes (as fast as within about ten seconds of Hh arrival) also accords with a Hh/Smo effect not mediated by transcription (Belgacem et al, ibid).

Since the transcriptional effects of Hh/Smo signaling are executed by Ci/Gli proteins in all examined species and the nucleotide sequences recognized by these transcription factors are very similar in different species and different Hh target genes, a straightforward first step in identifying particular Hh target genes would be to search for Ci/Gli binding sequences in the sequenced genomes. Gurdziel K et al (BMC Developmental Biology 2016; 16:4) reported results of such analyses. Human and mouse genome sequences and experimental determinations of promoter and enhancer sequences of numerous genes in human and mouse are known. Analyses of human and mouse genomes show that large numbers of Ci/Gli consensus sequences exist within and around the genes in both genomes. Chromatin immunoprecipitation of Ci/Gli binding genomic regions in a particular tissue or cell type, followed by analyses of the nucleotide sequences of precipitated DNA has been commonly used for determining functional Ci/Gli binding regions in that tissue or cell type (e.g. Vokes S A et al, Genes & Development 2008; 22:2651-2663). Such analyses have shown over 500 Ci/Gli binding regions and over 200 genes in vertebrates that are direct targets of Hh/Smo signaling even by the conservative estimates from testing in one tissue of an animal in a given time frame (Gurdziel et al, ibid; Vokes et al, ibid). Thus very large numbers of direct Hh target genes are determined even by non-inclusive screening assays. Different combinations of expressions of them can be calculated to be astronomical in number. In other words, a priori, huge numbers of different responses of different cells can be expected to Hh when they are presented with Hh.

Stimulation of proliferation of various cell types upon exposure to Hh was one of the earliest findings following the determination and cloning of Hh encoding genes. Inductions of differentiation of various cell types by Hh/Smo signaling were also commonly found. Following the earlier investigations with developing embryos, it soon became clear that Hh/Smo signaling is utilized through entire life in species from drosophila to human and that Hh/Smo signaling is absolutely required for vital normal functions and survival of adults (e.g. Hahn H et al, Journal of Biological Chemistry 1996; 271:12125-12128; Takabatake T et al, FEBS Letters 1997; 401:485-499; Traiffort E et al, European Journal of Neuroscience 1999; 11:3199-3214; Koebernick K et al, Mechanisms of Development 2001; 100:303-308; Sato N et al, Journal of Clinical Investigation 1999; 104:855-864; Van der Eerden B C et al, Journal of Bone and Mineral Research 2000; 15:1045-1055; Detmer K et al, Blood Cells Molecules and Diseases 2000; 26:360-372; Zhang Y et al, Nature 2001; 410:599-604). In particular normal stem cell functions are found to be absolutely depended on Hh/Smo signaling in adults (e.g. Zhang Y et al, ibid) and normal stem cell functions are known to be obligatory for continued survival of every person. Persons accidentally exposed to agents that harm stem cells reveal that whereas bone marrow stem cells can be supplied to such persons to save their life thanks to the relatively advanced state of the art in procurement and transplantation of marrow stem cells and blood, it is not possible to maintain the survival of victims in the long term when there are tissues, organs and systems besides the hematopoietic system that are also affected and whose stem cell functions are not provided (Densow D et al, Stem Cells 1997; 15, Supplement 2:287-297; Ishii T et al, Journal of Radiation Research 2001; 42:S167-S182; Pellegrini G et al, Transplantation 1999; 68:868-879). Such patients are found to die regardless of whether a victim may be kept alive in the short term with intensive medical care. Investigations in which Hh/Smo signaling is selectively inhibited even in a single tissue or organ confirm that Hh/Smo signaling is absolutely required for survival of adults (e.g. Zacharias W J et al, Gastroenterology 2010; 138:2368-2377).

Conditional genetic means of eliminating a protein required for Hh/Smo signaling represents an ultimate selective means of inhibition of the signaling. Since Smo is essential for transduction of the signaling initiated by Hh, Lavine K J et al (Journal of Clinical Investigation 2008; 118:2404-2414) used conditional genetic elimination of Smo in adults to observe its effects. They describe that selective inhibition of Hh/Smo signaling in adults, even only in heart, caused rapid demise and death of adult animals with dilated ventricles and other structural and functional indicators of heart failure.

Both transcriptional and non-transcriptional effects of Hh/Smo signaling are known to be required for numerous normal functions in adults. Sasaki N et al (Molecular and Cellular Neuroscience 2010; 45:335-344) describe examples of uses of the non-transcriptional effects in the central nervous system during postnatal life in postmitotic neurons and describe methods to identify the molecules that interact with Smo in cells. Because arrival of Hh to a cell or loss-of-function mutations of Patched causes activation of Hh/Smo signaling and Smo is essential for transduction of the signaling and for both transcriptional and non-transcriptional effects, effectors of Smo function have been widely examined and methods to identify molecules that can be used to inhibit (or to stimulate) Smo are known (e.g. Jiang K et al, Methods Mol Biol 2015; 1322:45-60; Sasaki N et al, ibid).

Affinity-purified and monoclonal function-blocking anti-Hh antibodies have been made and shown to provide selective inhibition of Hh/Smo signaling in the administered embryos by multiple criteria (e.g. Ericson J et al, Cell 1996; 87:661-673). The brain in the vertebrate embryos that has loss of Hh expression shows inhibition of differentiation of various neural cells that are normally induced by Hh and the animals show consequent brain malformations that include fusion of the developing eyes, called cyclopia (Krauss S et al, Cell 1993; 75:1431-1444). Causation of such brain malformations and cyclopia and deaths of fetuses and mothers in the animals administered with the teratogenic Veratrum alkaloids cyclopamine or jervine had been determined in various vertebrate species (Keeler R F, Proceedings Of The Society For Experimental Biology and Medicine 1975; 149:302-306; Omnell M L et al, Teratology 1990; 42:105-119). Incardona J et al (Development 1998; 125:3553-3562) and Cooper M K et al (Science 1998; 280:1603-1607), using methods like in the earlier investigations of Ericson et al (ibid), described that exposure of developing chicken embryos to cyclopamine or jervine caused these brain malformations and cyclopia due to a direct and selective inhibition of Hh/Smo signaling in the animals. Administration of cyclopamine or jervine to the developing embryos was found to cause a phenocopy of a Hh loss-of-function mutation and further test results showing a direct and selective inhibition of Hh/Smo signaling in the animals by these molecules have also been described (Incardona et al, ibid; Cooper et al, ibid).

Cyclopamine, a steroid alkaloid, has the chemical structure shown below.

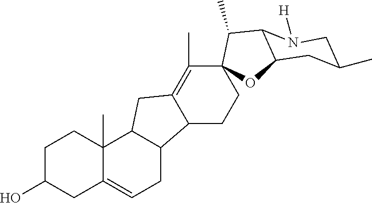

Selective inhibition of Hh/Smo signaling in animals by administration of cyclopamine has been described. Cyclopamine is found naturally in Veratrum plants and can be obtained by purification from this and other sources and can also be synthesized by methods of organic chemistry.

Automatable in vitro assays with a Gli recognition sequence-driven reporter have been described and provide quantitative data about the inhibition of Hh/Smo signaling by candidate compounds rapidly (e.g. Sasaki H et al, Development 1997; 124:1313-1322). Using patched −/− cells in such an assay, Taipale J et al (Nature 2000; 406:1005-1009) described that cyclopamine inhibits Hh/Smo signaling downstream of Patched, at the level of Smo, and described a derivative of it that was found to be more potent in the same assay. Molecules of interest determined to inhibit Hh/Smo signaling in such in vitro screens can then be tested in an available animal model for suitability for selective inhibition of Hh/Smo signaling in animals. Gaffield W et al (Cellular and Molecular Biology 1999; 45:579-588) described results of such animal testing and selective inhibition of Hh/Smo signaling in the administered chicken embryos by cyclopamine and enhancement of the inhibitory activity by conversion of cyclopamine to its 4-ene-3-one derivative.

Methods employing developing chicken and other embryos as convenient tools have been widely used for determining whether or not a molecule of interest can be used for selective inhibition of Hh/Smo signaling in animals. Stenkamp D L et al (Developmental Biology 2000; 220:238-252) and Nasevicius A et al (Nature Genetics 2000; 26:216-220) have described that developing zebrafish provide a particularly suitable model due to the ease of observation of the effects of administered molecules and known Hh loss-of-function mutants. They have described purpose made new molecules for selective inhibition of Hh/Smo signaling and causation of such inhibition in the administered animals by multiple criteria, including the phenocopying of a loss-of-function mutation of Hh and selective inhibition of differentiation of various cell types in vivo that are normally induced by Hh (Stenkamp et al, ibid; Nasevicius et al, ibid).

Studies of tumor cells from patients having tumors of various organs have revealed that a subset of the tumors show Hh/Smo signaling (e.g. Fujita E et al, Biochemical and Biophysical Research Communications 1997; 238:658-664; Reifenberger J et al, Cancer Research 1998; 58:1798-1803 and references therein). Quantitative analyses showed markedly greater Hh/Smo signaling activity in tumor cells than in the normal cells in the same patients (e.g. on average about 7× or greater in the case of basal cell carcinomas; Tojo M et al, Pathology International 1999; 49:687-694).

Predisposition to occurrences of some tumors by activation of Hh/Smo signaling was suggested also by the findings that nevoid basal cell carcinoma syndrome patients are born with a mutant patched allele in all cells to cause increase of Hh/Smo signaling activity due to the patched haploinsufficiency and that these patched+/−subjects develop basal cell carcinomas and certain other tumors as they grow older (Kimonis V E et al, American Journal of Medical Genetics 1997; 69:299-308). Animals engineered to have patched haploinsufficiency in all cells have also been found to show increased probability of occurrences of tumors of some organs as they grow older (Goodrich L V et al, Science 1997; 277:1109-1113; Aszterbaum et al, Nature Medicine 1999; 5:1285-1291). Goodrich et al, ibid, reported that medulloblastomas were observed in about 8% of patched+/−mice at 5 weeks of age and in about 30% of them at 12 to 25 weeks of age. Aszterbaum et al, ibid, reported increased occurrences of skin tumors in patched+/−mice in comparison to wild-type control animals with aging and exposure to agents that cause damage to the genetic material. With ultraviolet irradiation of skin, 3% of the 3-8 months old patched+/−mice were found to show tumors of skin and 40% of the patched +/−mice older than 9 months were found to have skin tumors. Such irradiation is known to cause damage to the genetic material and increased probability of occurrences of mutations and epigenetic changes throughout the genome.

Besides the loss-of-function mutations of patched, certain gain-of-function mutations of smo have also been described to cause activation of Hh/Smo signaling and found in some tumors (Xie J et al, Nature 1998; 391:90-92; Reifenberger J et al, Cancer Research 1998; 58:1798-1803). In accord with the above mentioned findings with subjects born with a Hh/Smo signaling activating mutation in all cells and found to develop tumors from a very small proportion of the cells with aging and exposure to mutagens, Xie et al, ibid, also reported insufficiency of a constitutive activation of Hh/Smo signaling for neoplastic transformation. In an in vitro assay of neoplastic transformation using known oncogenic viral gene controls, they reported that no transformed foci were observed in cells transfected with a gain-of-function mutant smo alone that caused constitutive activation of Hh/Smo signaling. Investigators using conditional genetic means of expression of smo mutants having gain-of-function (activating) mutations in experimental animals have also found insufficiency of such constitutive activation of Hh/Smo signaling for tumor occurrence (e.g. Migone F F et al, Genesis 2012; 50:28-40).

Aszterbaum et al (Nature Medicine 1999; 5:1285-1291) reported that tumor cells rendered devoid of Hh/Smo signaling showed slowing of proliferation during a period of 10 months of observation in culture. Taipale J et al (Nature 2000; 406:1005-1009) likewise reported a slowing of proliferation of other transformed cells that were rendered devoid of Hh/Smo signaling by treatment with a derivative of cyclopamine in vitro.

Extensive experience with treatments of tumor bearing human show that there are significant physiological-genetic differences of human from the experimental animal models commonly used in the early assessments of cancer chemotherapy candidates that make a successful therapeutic outcome in tumor bearing human relatively far difficult and that the vast majority of such candidates with a desirable effect e.g. in mice fail in human because of significant species differences (Takimoto C H, Clinical Cancer Research 2001; 7:229-230).

Various molecules described to provide selective inhibition of Hh/Smo signaling have been newly synthesized or purified from natural sources, incorporated to pharmaceutical formulations and administered to cancer patients for therapeutic purpose following the previous descriptions of use of a selective inhibitor of Hh/Smo signaling in treatment of tumor bearing human (Taş S et al, WO 02/078703). Patients having tumors of various organs have been treated with administrations of a medicament comprised of a selective inhibitor of Hh/Smo signaling and the treatments included combinations with other cancer chemotherapy (e.g. Jimeno A et al, Clinical Cancer Research 2013; 19:2766-2774; Goldman J et al, Clinical Cancer Research 2014; 21:1002-1009; Wagner A J et al, Clinical Cancer Research 2014; 21:1044-1051; Sasaki K et al, Leukemia & Lymphoma 2015; 56:2092-2097; Minami H et al, Cancer Science 2016; 107:1477-1483 and references therein). In the investigations with a selective inhibitor of Hh/Smo signaling without accompanying other cancer chemotherapy the results showed disease stabilization or a slowing of the rate of tumor growth in a subpopulation of patients and more infrequently tumor shrinkage with subsequent regrowth, and rarely radiographic undetectability of tumor that was assumed as tumor disappearance but was disproven with subsequent relapse of tumor (Jimeno et al, ibid; Goldman et al, ibid; Wagner et al, ibid; Sasaki K et al, ibid; Minami et al, ibid, and references therein). The relapsed tumoral disease in the patients was found to be generally treatment resistant.

### SUMMARY OF THE INVENTION

This invention concerns drug treatments of cancer and other pathological conditions associated with aging.

In one aspect, the invention concerns treatment of tumor bearing human by use of a medicament comprised of a selective inhibitor of Hedgehog/Smoothened signaling for elimination of tumor cells from patient independent of the histopathological class and stage of tumor, including the metastatic. The described tumor treatment provides disappearance of tumor from patient without recurrence and without causation of damage to the genetic material of treated patient. Sparing of the normal cells of treated patients that include those that are dependent on Hedgehog/Smoothened signaling for normal functions adds to the advantages of the treatment.

In a further aspect, methods to decrease damaging of the genetic material with aging and therapeutic utility thereof are described.

### BRIEF DESCRIPTION OF THE FIGURES

This patent application file contains, besides figures printed with black ink, figures in color because immunohistochemical data and in vivo images of tissues, due to their nature, can be presented best in the form of color photographs.

FIG. 1 shows photograph of a section of tumor bearing tissue and shows that the described tumor treatment that induces differentiation of tumor cells prior to their apoptosis provides sparing of normal cells of patient, including the normal stem cells and multipotent progenitors. In this example tumor bearing skin was excised after topical applications of a selective inhibitor of Hh/Smo signaling and sections were immunohistochemically stained with a primary antibody that detects Ep-CAM which is a protein expressed both by untreated tumor cells and by normal stem cells and progenitors and rapidly disappears upon differentiation of them. The cells binding the anti Ep-CAM antibody (monoclonal antibody Ber-EP4) were visualized by peroxidase staining (brown). A hair follicle is seen on right with brown colored normal epithelial cells that were exposed to the same dose of cyclopamine as the adjacent tumor cells that show absence of detectable Ep-CAM expression. As the untreated tumor cells consistently show intense staining with the anti Ep-CAM antibody, normal stem and progenitors are seen to have been spared following exposure to a dose of a selective inhibitor of Hh/Smo signaling sufficing for induction of differentiation of all tumor cells.

FIG. 2 shows photograph of a section of a tumor that was excised after causation of shrinkage of it by treatment before it disappeared and shows that the efficient apoptotic removal of tumor cells from patient by the described tumor treatment is preceded by induction of differentiation of tumor cells. This section is from a tumor excised on second day of administrations of a selective inhibitor of Hh/Smo signaling. The section was immunohistochemically stained (brown, with peroxidase) using a primary antibody that binds to a protein that shows increase of expression with differentiation of the tumor cells (CD44 standard in this example). The tumor cells displaying characteristic morphological signs of apoptosis such as rounded dark nucleus are seen to show brown colored periphery of cytoplasm, pointing to induction of differentiation of them prior to apoptosis. Empty spaces devoid of tumor cells are seen to be forming in the tumor as a result of the efficiency of removal therefrom of tumor cells by induction of apoptosis of them.

FIG. 3A shows photograph of a tumor grown into the lumen of trachea near tracheal bifurcation. Photograph was taken during bronchoscopic examination of the lung tumor and respiratory airways prior to the initiation of treatment.

FIG. 3B shows photograph of the same tumor as in FIG. 3A soon after the direct injection of medicament into it in the first session of medicament administrations. Slight bleeding from the tumor due to such administration is seen.

FIG. 3C shows photograph of the same tumor as in FIGS. 3A and 3B on the fourth day of treatment. The photograph was taken before the start of the injections in the third session of treatment on day four. Marked decrease of size of the tumor relative to the pre-treatment size is seen. Normal tissues around the tumor exposed to the medicament do not show a sign of harming. A small hematoma in the shrinked tumor is visible.

### COLOR PRINTS

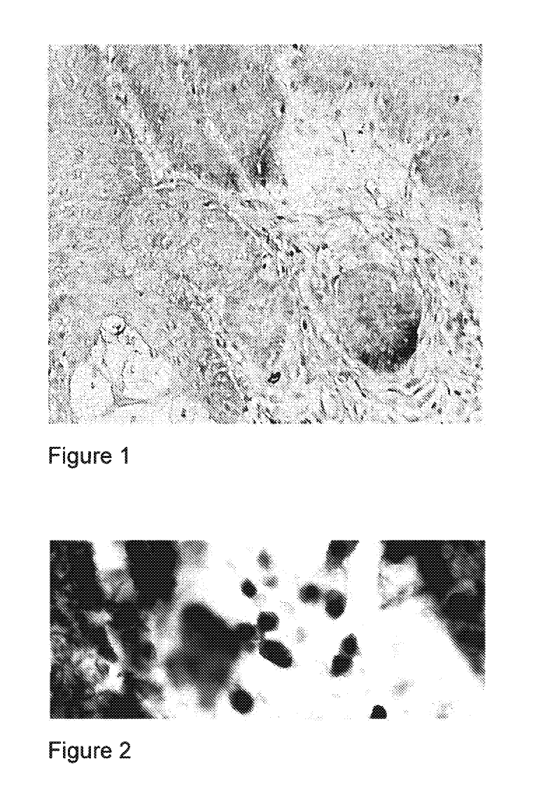

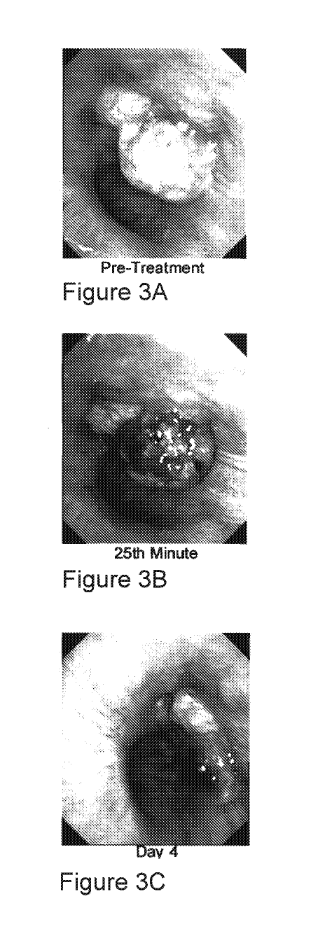

This patent application file contains, besides figures in gray scale, figures in color because immunohistochemical data and in vivo images of tissues, due to their nature, can be presented best in the form of color photographs. Color prints of the same figures as on pages 1/2 (FIG. 1, FIG. 2) and 2/2 (FIG. 3A, FIG. 3B, FIG. 3C), are provided in this application file as pages 1/2a and 2/2a, respectively, for that reason. Consideration of the above mentioned fact by the Patent Authority and the keeping of pages 1/2a and 2/2a as part of this patent application is requested. However, pages 1/2a and 2/2a may be removed from the patent application file if it is deemed necessary by the Patent Authority.

### DETAILED DESCRIPTION OF THE INVENTION

This invention concerns rational therapeutic interventions with the pathological conditions associated with aging, including cancer.

Previous investigations with cell nuclei and chromatin prepared from liver of young adult and old mice had revealed increase of alkali-labile DNA lesions and strand breaks with increasing age of animals as determined by hyperchromicity measurements of DNA under the influence of alkaline pH with monitoring of turbidity in the presence and absence of a disulfide reducing agent (Taş S et al, Gerontologist, Part II, 1978; 18:131). Alkali-labile DNA lesions are known to include apurinic and apyrimidinic sites. Density gradient centrifugation analyses of demembranized cell nuclei and of chromatin through neutral sucrose gradients in the presence and absence of a disulfide reducing agent also revealed occurrences of such damage and alterations of chromatin structure with aging when they were prepared from liver and from mononuclear spleen cells of mice (Taş S et al, Mechanisms of Ageing and Development 1980; 12:65-80; Taş S et al, Mechanisms of Ageing and Development 1982; 19:73-84). I have subsequently determined likewise donor age associated increased occurrences of DNA damage and chromatin structure alterations with demembranized nuclei and chromatin prepared from mononuclear blood cells of human when they were analysed by the aforementioned methods used with the preparations from mice and also by agarose gel electrophoretic analyses in the presence and absence of sodium dodecyl sulfate and/or a disulfide reducing agent and/or NaOH for alkalinisation of pH. Gel electrophoretic separations of DNA molecules with resolution beyond conventional size limits and simultaneous determinations of both nucleic acid and protein components of chromatin in the same gel lane facilitate chromatin structure analyses (Taş S, Analytical Biochemistry 1990; 188:33-37). Demebranizing intact cell nuclei with a non-ionic detergent and placing in gel or on top of a density gradient, followed by electrophoresis or centrifugation respectively, facilitates analyses that avoid artefactual breakages of large DNA fragments (e.g. due to pipetting shear) and analyses of supercoiling of DNA (supercoiled by the looping of DNA in cell nucleus akin to the supercoiling of the circular prokaryotic DNA) by use of ethidium bromide allow detection of even a single single strand breakage in the looped DNA (Taş et al 1982, ibid; Taş S et al, Cytologia 1985; 50:405-415).

The eukaryotic genetic material's structure enables differential expressions of the genetic information in a heritable manner without a necessary change of the nucleotide sequence. Specifically, there are chromatin structure changes that enable such differential expressions of the genetic information and they are absolutely required for the cellular differentiation that makes higher multicellular eukaryotes possible. On the other hand, the chromatin structure changes required for cellular differentiation, in particular the heterochromatinization, come with a cost, including in the restriction of the repair of particular forms of damage to the genetic material, and they can be determinants of biological aging (Taş S, Monographs In Developmental Biology 1984; 17:178-192).

Human is an advanced multicellular eukaryote with the longest maximum lifespan potential among hominids. Comparisons among mammalian species show that increase of maximum lifespan potential is significantly correlated with improved maintenance of genomic integrity (e.g. Grube K et al, Proc Natl Acad Sci USA 1992; 89:1159-1163). Despite the relatively more effective prevention of oxidative and other damage to the genetic material and improved repair of such damage in human, above mentioned test results show that human is also faced with increased occurrences of such damage during aging. The age associated increases of unrepaired-misrepaired damages of genetic material and of somatic mutations are not inconsequential and they occur both in postmitotic cells (e.g. neurons in brain and muscle cells in heart) and in the cells that are capable of proliferating. For example neuronal losses and nervous system malfunctioning in senile dementia-Alzheimer's disease are found to be associated with such damage in neurons. A mutation because of unrepaired-misrepaired damage of genetic material may have serious or minor functional effect depending on its nature and the cell involved. Mutations in stem cells are in general dangerous not the least because the subsequent progeny of such long lived cells also show them. I have also found that structural alterations of chromatin that are obligatory for cellular differentiation in eukaryotic organisms (heterochromatinization) and the oxidative metabolism in mitochondria that generates ATP in far greater quantities than that achieved by nonoxidative metabolism are sources of increase of unrepaired-misrepaired damage and mutations in human with aging. Hence these inherent features of eukaryotes and of human organism create dilemmas and unsolved problems concerning treatments of the pathological conditions accompanying aging.

Hh/Smo signaling has been described to induce differentiation of various cell types and is conserved in species from drosophila to human implying indispensability. The signaling gets more complex in human [e.g. the presence of three homologs in human (Shh, Ihh, Dhh) of the single Hh of drosophila] but its basic features are similar in different species. Nucleotide sequences of the genes encoding for the proteins that participate in Hh/Smo signaling and the amino acid sequences of encoded proteins are known for human and several other species (references cited in relation to Hh/Smo signaling under background of the invention have references of these). Requirement of Hh/Smo signaling for vital normal functions throughout human body, very large number of Hh target genes and the significant physiological differences of human from other species make it necessary to determine effects of a use of a regulator of the signaling in human in vivo although investigations in vitro and in other species may be informative. Using an experimental design that allows simultaneous determinations of the effects of varying doses of a selective inhibitor of Hh/Smo signaling on the various different cell types in a tissue in vivo, I have determined effects of Hh/Smo signaling in health and in pathogenesis of age associated diseases in human.

Cyclopamine is a hydrophobic small molecule that can be used for selective inhibition of Hh/Smo signaling. It has little solubility in aqueous media but can be solubilized in ethanol and can also be bound to human serum albumin and other plasma proteins and lipoproteins that are soluble in the extracellular fluids in human. These cyclopamine binding proteins and lipoproteins can transport cyclopamine through aqueous media in blood plasma and extracellular fluids. Ethanol is continuously miscible with water and body fluids. Dissolving cyclopamine in ethanol and incorporating into a cream or gel formulation and applying such onto e.g. skin surface in measured quantities allows generation of concentration gradients of cyclopamine in tissue, in this example in the skin tissue. Cyclopamine applied to skin in this way diffuses from the site of application vertically through epidermal layers and dermis besides showing some horizontal diffusion. The applied skin sites can be observed with naked eye and dermoscopy for effects detectable by such and biopsies can be taken after varying periods from one or repeated applications. Punch biopsies and other methods of obtaining skin specimens can be used. The specimens obtained as above (along with those from appropriate control, e.g. placebo applied skin) are then processed for histopathological and other examinations. Following fixation, embedding and sectioning, sections can be stained by H&E and other stains. Immunohistochemical staining of serial sections for detections of molecules of interest in the analysed tissue are particularly informative. Freezing a part of the freshly obtained tissue is useful for in situ hybridization analyses of expressions of particular RNA molecules of interest and for quantitative measurements, e.g. by those based on the polymerase chain reaction (PCR). Frozen tissue can also be used for other molecular analyses, including for small molecule metabolites.

Examinations of serially sectioned tissue specimens by immunohistochemical methods for effects on expressions of Hh target genes (e.g. Patched, Gli1, Gli2, Gli3) and supplementing these with in situ hybridization analyses (e.g. where a primary antibody is not available) can provide a readout of the effects of varying concentrations of cyclopamine on different cell types and tissue structures in their natural environment in vivo. Additional methods of tracking the diffusion and effects of a selective inhibitor of Hh/Smo signaling applied as above that can also be used (e.g. imaging by use of a radiolabelled tracer) were not needed in the investigations described here since the above mentioned examinations gave unambiguous results as described below.

Cyclopamine can be incorporated into various cream, gel and other topical formulations. In the examples below a base comprised of heavy paraffin oil (10% w/w), vaseline (10% w/w), stearyl or cetyl alcohol (8 to 10% w/w), polyoxylsteareth-40 (3 to 6%) with the remainder being ethanol and water were used with similar results and hydroxypropylmethyl cellulose (average molecular mass about 800000) was added to 1.5% w/w for preparation of a gel. Final concentration of cyclopamine in the cream and gel formulations used was 18 mM.

The first-on-human testing of a pharmaceutical composition comprised of cyclopamine was carried out on healthy normal skin. Effects on the applied skin were observed and on the basis of them the investigations described in Taş S et al, PCT/TR01/00027 (published as WO 02/078703) were carried out with tumor bearing human. These investigations have shown that beneficial therapeutic effects can be caused in tumor bearing human when a selective inhibitor of Hh/Smo signaling is used as described.

Large numbers of men and women having tumors of various organs have been reported to have been treated by administrations of medicaments comprised of a selective inhibitor of Hh/Smo signaling in several clinical trials after the earlier descriptions in Taş S et al, WO 02/078703 (e.g. Jimeno A et al, Clinical Cancer Research 2013; 19:2766-2774; Goldman J et al, Clinical Cancer Research 2014; 21:1002-1009; Wagner A J et al, Clinical Cancer Research 2014; 21:1044-1051; Sasaki K et al, Leukemia & Lymphoma 2015; 56:2092-2097; Minami H et al, Cancer Science 2016; 107:1477-1483 and references therein). Causation of a slowing of tumor growth or disease stabilization have been reported in a minority of these patients by the described treatments. Causation of tumor shrinkage was found in a smaller subpopulation of them. These patients, and a still smaller subpopulation in whom disappearance of a tumor was presumed, were found to show subsequent regrowth of tumor. The regrowths of tumor were found to be generally unresponsive to attempts of treatment. In view of these results, combinations of administration of a selective inhibitor of Hh/Smo signaling with radiotherapy and/or another drug treatment of cancer for improvement of survival of patients have been discussed in the reports and reviews of the findings of these clinical trials.

Considering the record in the field of cancer chemotherapy after my previous descriptions concerning use of a selective inhibitor of Hh/Smo signaling for treatment of tumor bearing human, I have analysed the reported treatments of cancer patients in the published trials and carried out new investigations in this field. I describe here my findings and describe drug treatments that provide disappearance of tumor in patients without recurrence and provide also avoidance of causation of new tumors.

Intranuclear executers of Hh/Smo signaling, the Ci/Gli transcription factors, act by recognizing and binding to their consensus sequences in Hh target genes. Chromatin structure of a particular Hh target gene in a cell creates a further level of variability of the responses of different cells in a tissue to Hh and to a selective inhibitor of Hh signaling on top of the variability caused by the large numbers of Hh target genes and their regulation by enhancers that can bind multiple transcription factors to make a particular gene's responses affected by other signals as well in a cell. The experimental design that I mentioned above allows simultaneous determinations of the responses of different cell types in a tissue to varying doses of a selective inhibitor of Hh/Smo signaling in vivo. With that experimental approach and by analyses of expressions of molecular markers of proliferating, differentiating and apoptosing cells in tissues together with morphological criteria, I determined that whereas topical applications of e.g. 18 mM cyclopamine as described above onto tumor bearing skin of patients every 4 hours until about 25 micromole cyclopamine per square centimeter skin surface cumulation could produce shrinkage and even disappearance of a tumor to naked eye depending on its size and depth of growth into dermis, occasional undifferentiated tumor cells (identified e.g. by their EpCAM expression) could remain at deeper invading fronts and be detected by microscopic examination.

Whether or not a tumor is caused to disappear without recurrence makes a critical difference for tumor bearing patients and whether or not a given treatment has costs on the health of patient is also pertinent as the safety profile of a treatment is an element of therapeutic efficacy. I made determinations about them.

Because numerous varying molecular signals affect proliferation, differentiation and survival of cells, whether normal or neoplastic, and many interactions between cells in tissues in vivo are not reproduced in vitro, I investigated the effects of Hh/Smo signaling on them in vivo by the above described experimental arrangement. Results of quantitative determination of the effects on proliferation and survival of tumor cells following their exposure to varying doses of cyclopamine in vivo as determined by such investigations have shown that (1) a selective inhibitor of Hh/Smo signaling can be used for inducing apoptosis of tumor cells in a patient, (2) induction of apoptosis is depended on the dose of the selective inhibitor of Hh/Smo signaling to which tumor cells are exposed, and (3) the lowest dose sufficient to induce apoptosis of the tumor cells is significantly greater than that suffices for inhibition of proliferation of the same tumor cells. A further pertinent finding in these investigations has been that normal cells, including the normal stem cells and multipotent progenitors are spared following exposure to a dose of a selective inhibitor of Hh/Smo signaling that causes differentiation of all adjacent tumor cells. FIG. 1 shows an example. Similar results were found with analyses of sections of different tumors and with further molecular markers of stem cells and of their progeny.

Parallel examinations of serial sections of tumor bearing tissue for markers of proliferating and apoptosing cells revealed further that exposure of tumor cells to a selective inhibitor of Hh/Smo signaling in a dose above that suffices for induction of differentiation of them causes apoptosis of them as shown in the example in FIG. 2. Molecular markers of differentiation in a lineage of cells in each organ are in general known and methods of their detection, such as immunohistochemistry, can be used to determine cellular differentiation. A molecular marker of differentiation may be one that is expressed or one that disappears upon differentiation of a particular cell type. Ep-CAM is an adhesion molecule that is expressed by normal stem cells and multipotent progenitors and rapidly disappears with differentiation to their downstream progeny (e.g. Anderson R et al, Journal of Reproduction and Fertility 1999; 116:379-384; DeBoer C J et al, Journal of Pathology 1999; 188:201-206). It is found to be expressed also by most cancer cells and various monoclonal antibodies that recognize Ep-CAM are known (e.g. Ber-EP4).

In the example of a case where long-term monitoring of the patient was feasible, a basal cell carcinoma on face measuring about 3×4 mm on surface was administered with about 15 μl of a cream preparation having 18 mM cyclopamine every 6 hours and the administrations were discontinued when the tumor had become invisible to the naked eye after the fourth day. Follow up in this case showed a tumoral growth at the same site of skin after about 5 months. Because cyclopamine or another selective inhibitor of Hh/Smo signaling and facilities for preparing a medicament were not available in this case at the time of the recurrence, several months elapsed until such could be arranged and the tumor reached to about 1 mm larger size on both axes than that at the time of the above mentioned first treatment when the treatment described below was initiated.

A cream containing 18 mM cyclopamine and 10% urea (w/w) was prepared similar to that described above. Urea is a naturally occurring product of amino acid catabolism in human. At concentrations around the concentration that was used it facilitates penetration of cyclopamine through stratum corneum and epidermis to the tumoral growth in dermis. About 25 μl of this cream was applied onto the surface of tumor bearing skin every 6 hours. The tumor started to show signs of regression on day 3 of the treatment and showed visible decrease of its height from skin surface on day 5 of treatment. With continuation of these medicament administrations every 6 hours, the tumor became not recognizable by naked eye on day 8. Inspection of skin did not show a detectable adverse effect. The same administrations were continued for four more days until discontinuation on day 12 at which time no tumor was detectable and no adverse effects were found in the skin region onto which the administrations were made. Weekly monitoring during the next three weeks and approximately monthly checks for the following several months also showed absence of a detectable adverse effect and absence of tumor. Causation of disappearance of the tumor without recurrence was verified about 3½ years later.

FIG. 3A shows photograph of a tumor extending into the tracheal lumen in a man prior to treatment. Imaging of the patient by computed tomography, PET scanning and other imaging modalities showed a tumor occupying the superior and middle lobes and upper segment of inferior lobe of right lung. The tumor showed growths also to the outside of lung. Multiple lymph nodes in the mediastinum were involved. Right hilar region was nearly completely occupied and right pulmonary artery was surrounded by it. The tumor extended to the superior mediastinum and infracarinal regions and showed also signs of distant metastasis in whole body imaging. Histopathological investigations of bronchioalveolar lavage cells and of a biopsy obtained by bronchoscopy revealed adenosquamous carcinoma of lung. Patient had become severely dyspneic and bed-bound. Attending thoracic surgeon and physicians had concluded that surgical excision of tumor was not a therapeutic possibility and that pain killers may be used to help him to cope with his situation. Patient's and his family's application for treatment by the instant treatment was evaluated and accepted. Repeats of the pathological and other laboratory and clinical examinations confirmed the previous diagnosis of malignant disease. Bronchoscopy and imaging revealed obstruction of the superior lobe's bronchus, involvements of other bronchi and narrowing of the tracheal lumen by the tumor. The photograph in FIG. 3A was taken near tracheal bifurcation during the bronchoscopic visualizations. The tumor of lung was estimated to have a volume of about 245 cubic centimeter by magnetic resonance imaging.

In view of the poor clinical status and weakness of patient and the severe dyspnea associated with the obstruction and narrowing of the large airways, a two stage treatment strategy aiming to provide first adequate breathing and improvement of his general condition was designed with administrations of a selective inhibitor of Hh/Smo signaling in light of the previous findings in tumor bearing patients whose tissue samples were analysed following varying exposure to such an inhibitor. Cyclopamine was used to prepare a medicament comprised of a selective inhibitor of Hh/Smo signaling for the below described treatment not because cyclopamine is an optimal molecule for use in the instant tumor treatment as pointed out later, but because it was the only selective inhibitor of Hh/Smo signalling that was available for this patient at the time of diagnosis. Under general anesthesia a medicament comprised of 18 mM cyclopamine in 98% ethanol, 2% phosphate buffered saline pH 7.4 was administered by direct injections into the tumoral growths into trachea and right lung's large airways with the aid of a bronchoscope. The medicament solution had been sterile filtered through a 0.2 μm pore size filter. A needle having 1.2 cm length was inserted to tumor to about 1 cm depth and about 2 ml of the medicament solution was administered during a period of about 5 minutes while slowly withdrawing the needle from the depth to which it was inserted. The endoscopist was given latitude for injection around that rate of injection. Optimal number of the distances between each injection site varies depending on the configuration and size of a tumoral growth and optimal rate of injection can also vary depending on a particular tumor and interstitial fluid pressure in a tumor. Ideally an injection pump allowing accurate adjustment of rate of injection is preferred and an additional line joining the tubing near the needle and allowing co-administration of an appropriate diluent so that the concentration of ethanol exiting the needle can be reduced is preferred. Ethanol at high concentrations (e.g. absolute or 98%) is known to cause lysis of cell membranes to cause necrosis and to cause denaturation-precipitation of many proteins and direct injections of such concentrations of ethanol have long been used for causation of necrosis of small tumors by direct injection. Ethanol is readily miscible with aqueous media and can also help convection-enhanced delivery of drug molecules solubilized in it when intratumorally injected. Injection of a medicament solution having high concentrations of ethanol at slow enough rates can provide significant dilution of the small droplets of the ethanol carrier exiting the needle tip by the interstitial fluid in tumor. In the present example the tumoral growths into the airways were injected at positions about 2 to 3 cm apart under bronchoscopic visualization as above while slowly withdrawing the needle from the site of insertion and sterile saline administrations were used as needed, including for control of bleeding from a site of injection. In general the bleedings were minor and spontaneously ceased and instillation of cold saline was used for control of bleeding for a site showing continued bleeding. FIG. 3B shows photograph of the intratracheal portion of the tumor following intratumoral injection.

Medicament administrations with the aid of a bronchoscope can be repeated in multiple sessions under anesthesia. In this case about 12 to 18 ml of 18 mM cyclopamine solution was injected directly into tumor in a session as above and was also instilled to small airways along with saline. About 48 hours after the first session, the medicament administrations were repeated in a second session as above. The tumor sites injected in the first session were seen to show significant decrease of size relative to the pre-treatment size when visualized during the second session. On the fourth day after the first session, a third session of bronchoscopic visualization and medicament administrations were repeated as above. On the fourth day the tumor had become markedly reduced in size and the formerly obliterated right bronchus had opened. Following the described intratumoral injections, the patient expressed ease of breathing. His physical examination and tests also showed improved respiration and showed lack of an adverse effect of the treatment. FIG. 3C shows the tumoral growth into trachea photographed on the fourth day at the start of the third session. It shows marked shrinkage of the tumoral growth into the tracheal lumen and the normal tissues around the tumor show no sign of an adverse effect.

The patient showed continued improvement of respiration and clinical status following the third session of medicament administrations. He was no longer bed bound and could walk and climb stairs without help. A magnetic resonance imaging on the eighth day of the start of medicament administrations showed that the lung tumor had decreased to about 45% of the pre-treatment size and there were no signs of an adverse effect of the medicament administrations in mediastinal structures. Tumor shrinkage at distances several centimeters away from the about 1 cm inserted needle tip, the distances at which ethanol concentration would be reduced to 5% and less even if it would not be diluted by means other than a simple diffusion through that distance, showed that the therapeutic effect was due to the dose of the selective inhibitor of Hh/Smo signalling reaching there. Cyclopamine can associate with albumin, lipoproteins and other tissue molecules for movements in tissues. With these results and continued improvement of the clinical status of patient, objective of the first stage of his treatment was considered achieved for proceeding to the next stage of treatment.

The removal of lung cancer cells from patient by exposing them to a dose of a selective inhibitor of Hh/Smo signaling that did not cause a detectable adverse effect in the normal bronchopulmonary tissue, neither by visual inspection (e.g. FIG. 3C) nor by functional criteria as attested by the improved (not worsened) respiratory functions following above described drug treatment, confirmed the likewise findings in patients with other tumors who were examined and whose specimens were analyzed. There was no evidence of causation of tissue necrosis, neither by bronchoscopic visualization of the medicament administered bronchopulmonary tissue, nor by blood chemistry determinations known to be able to reveal such occurrence. The findings in the tumor bearing bronchopulmonary tissue accorded rather with the removal of tumor cells from there by induction of apoptosis of them like that in the case of tumor bearing skin tissue.

The described technology for removal of tumor cells from tumor bearing human by administration of a medicament comprised of a selective inhibitor of Hh/Smo signalling while sparing the normal cells and normal functions of patient is advantageous for patients when a tumor's histopathological class and other features reveal that another treatment does not solve the problem of patient and/or when it is known to cause serious adverse effects in the normal cells of patient, including genotoxicity. Endoscopic means of visualizing various internal organs besides the lung are known and means of delivery of various solutions to an organ or tissue site during endoscopic visualization (e.g. for diagnostic purposes) are also known. Such tools can be used for delivery of a medicament comprised of a selective inhibitor of Hh/Smo signaling to another tumor bearing internal organ as exemplified for lung cancer. In addition various surgical procedures, including open surgery, can be performed to access an internal organ and tissue site to administer a medicament comprised of a selective inhibitor of Hh/Smo signaling thereto in a dose that provides apoptotic removal of the tumor cells while sparing the normal cells and functions of patient similar to that described here by use of a bronchoscopic device.

Additional investigations of tumor bearing patients and of tissue specimens that were analyzed as above after administrations of cyclopamine showed that induction of apoptosis of tumor cells while sparing normal cells is achievable only within an exposure window to a selective inhibitor of Hh/Smo signaling and that exceeding it can cause differentiation and apoptosis of normal cells as well. The above described experimental design and investigations involving administrations of varying quantities of cyclopamine for varying durations followed by tissue sampling for microscopic examinations allow additional determinations as well on the normal cells and tumor cells in vivo. When effects of equal estimated exposures resulting from a relatively longer exposure to a lower concentration of cyclopamine were compared with those resulting from a relatively shorter exposure to a higher concentration of cyclopamine, they were found to be different. To a first approximation, exposing the tissues to a relatively lower concentration of cyclopamine with increase of duration of exposure that might be considered safer was determined not to be so in consideration of the effects on normal cells and on tumor cells, including with regard to the risk of residual undifferentiated tumor cells.

Patients in whom a tumor has invaded one or more vital structure to prohibit a surgical intervention and tumors that have made metastases and micrometastases that can cause tumor recurrence pose a different problem from others. A patient having such a tumor is known to die due to the tumoral disease unless a systemic treatment is known that can be used to eliminate the tumor from the patient. Yet Hh/Smo signaling is found to be utilized throughout human body for multiple normal functions that are necessary for survival of every person. Not only the transcriptional effects of Hh/Smo signaling but also non-transcriptional effects of Hh/Smo signaling are found to be utilized for normal functions throughout organs and tissues and not only in cells with proliferation capacity but also in postmitotic cells and in organs that require them for normal functioning. Brain and heart represent two of such organs. Hh/Smo signaling in these organs are conserved across species and utilized e.g. in the central nervous system at vital sites in adults, including those involved in regulations of cardiovascular, respiratory and endocrine functions (e.g. Traiffort E et al, European Journal of Neuroscience 1999; 11:3199-3214). Causation of reduction of Hh/Smo signaling below a critical level by a systemically administered selective inhibitor of Hh/Smo signaling, for example at a central nervous site, can be prohibitive for elimination of a tumor from such a patient. Likewise causation of an adverse effect by a selective inhibitor of Hh/Smo signaling e.g. in cardiovascular system following a systemic administration for a tumor not possible to eliminate from the patient by different means can also preclude elimination of the tumor. A priori, effects in one or more of numerous other normal functions and organs that are depended on Hh/Smo signaling in adults can also preclude such therapeutic result. Patients at older ages are in this respect known to be generally vulnerable to increased demands on normal functions. For example “senile heart disease” is an entity called so because of occurrence predominantly in older adults and may manifest itself as congestive heart failure following a pneumonia that in younger adults is typically without such effect. Histopathology in autopsied cases shows diffuse myocardial fibrosis in the absence of myocardial infarction and shows lipofuscin deposition in the myocardial cells in testimony of age-associated damage in these postmitotic cells.

Cyclopamine has little solubility in ordinary aqueous media. It can be solubilized in ethanol for preparation of a medicament for use in the present drug treatment and other means of solubilisation and delivery of such a drug molecule can also be used (e.g. complexing with albumin or another physiological macromolecule that has the advantage of decreasing losses of pharmaceutically active molecule through glomerular filtration before reaching to the environs of the target tumor cells via systemic circulation). In the case of a medicament comprised of cyclopamine solubilized in ethanol for systemic administration, the rate of infusion should be adjusted by taking into account the actions of the ethanol carrier as well. Ethanol normally forms in small amounts in every person. Amounts of the ethanol solvent to be administered for treatment of a patient having a metastatic tumor can however be large and toxicity by it must be avoided as follows. Ethanol is frequently consumed by adults for its sedating and other effects and patients can show variation in their ethanol metabolism (same mg/kg/day amount of ethanol administered to different persons can cause varying effects depending on e.g. whether a person is chronic alcohol drinker or non-drinker). In general up to about 11 mM blood ethanol concentration can be sedative, 11-33 mM can cause decrease or lack of motor coordination, 33-43 mM can cause reversible ethanol intoxication and blood concentrations more than about 70-80 mM can cause unconsciousness and ethanol can be fatal at still higher concentrations. Ethanol is however metabolized rapidly so that by adjusting the rate of infusion one can achieve adequate systemic dosing of a patient with cyclopamine solubilized in ethanol without causation of intolerable effects of ethanol in the patient. Blood ethanol concentrations can be monitored by known methods (including indirectly through measurements in breath) and typically what mg/kg ethanol administrations produce what blood concentrations are also known. The above mentioned ethanol effects can be used as a guide for not exceeding an ethanol concentration in blood that would be intolerable.

Various means of non-oral systemic administration of medicaments have been known. Infusion into a vein is frequently practiced and other means of non-oral systemic administration are also known (e.g. administration to peritoneal cavity with aid of a catheter for passage from there to the systemic circulation). Since ethanol at high concentrations (e.g. 98% or absolute ethanol) can cause lysis of plasma membrane of cells and precipitation of proteins, its rate of entry into a vein or peritoneal cavity must be slow enough to provide dilution to avoid such unwanted effects. Administration by use of a Y shaped catheter arrangement where one line provides the cyclopamine-ethanol solution, the other provides an aqueous diluting solution (e.g. saline) and the two are mixed just before entry into vein or peritoneal cavity can be practiced to dilute the ethanol concentration to about 5-10% (or lower). Rate of infusion of a solution form medicament containing cyclopamine (e.g. 18 mM cyclopamine in 98% ethanol) can be adjusted by taking into account the effects of the carrier as mentioned above.

The treatment for a surgically unresectable tumor should be performed without delay as delaying can risk a patient's life despite an elimination of all tumor cells when the tumor growth until then causes critical organ damage. In the case of above described lung cancer patient continuing with systemic treatment soon after the improvement of his medical status was precluded for nonmedical reasons. His respiratory difficulties recurred in association with radiographically detected regrowth of the residual tumor during an interval of about 2½ months from the last day of the intratumoral medicament administrations until a hospital and other facilities for medicament preparation and treatment of the patient could be arranged. On the first day of systemic medicament administration the patient did not have a medical complaint other than about breathing. With the patient in recumbent position with elevation of head, a sterile solution comprised of 18 mM cyclopamine in 98% ethanol, 2% phosphate buffered saline pH 7.4 was administered by controlled rate systemic infusion with monitoring in light of the previous findings of the administrations of varying doses of cyclopamine by other routes. Patient's heart rate, blood pressure and other monitored functions did not show a significant change during an approximately constant rate infusion for about 9 hours and slowing afterwards that was estimated to deliver a dose of cyclopamine below 15 mg/kg/day. The patient expressed improvement of breathing starting in the evening of second day and was not observed to show an adverse effect other than nausea-vomiting controllable by antiemetic and visual hallucinations on two occasions. Laboratory analyses of blood, urine and feces samples on the first, second and third days and other investigations did not show causation of an organ damage and revealed highly efficient apoptotic removal of the tumor cells from him as discussed below with examples of investigation results.

In a patient having a large tumor load as in this patient, causation of rapid elimination of the tumor cells by induction of apoptosis of them, while advantageous, makes demands. Nucleic acids of tumor cells induced to undergo apoptosis are catabolized to uric acid which has limited solubility in aqueous media. Increased production of uric acid to cause blood concentrations above the solubility limit leads to precipitation of crystals of it that can initiate tissue damage. Measures for reducing elevation of uric acid in blood plasma and management of the patients having increased concentrations of uric acid in body fluids are known. Purines are catabolized to uric acid via AMP, which is deaminated to IMP which in turn is converted to hypoxanthine. Allopurinol causes inhibition of xanthine oxidase that converts hypoxanthine to xanthine before it becomes uric acid and can be used to decrease uric acid formation and precipitation. In combination with appropriate hydration of patient and diuretics, allopurinol can serve to decrease uric acid in blood plasma and to help its excretion without precipitation in renal tubules. Treatment of hyperuricemic patients with urate oxidase has been found to provide faster and greater reduction of blood uric acid than allopurinol and can also provide more effective prevention of occurrence of hyperuricemia than allopurinol. It provides breakdown of existing uric acid to water soluble allantoin that can be readily excreted with urine. Further measures for patients having hyperuricemia are also known and can also be used.

Myocardium's predominant use of the oxidative energy metabolism for its needs of ATP for the continuous pumping of blood also demands attention in treating a patient by systemic administration of a medicament comprised of a selective inhibitor of Hh/Smo signaling for elimination of tumor cells from the patient. Fatty acids, acetoacetate and glucose serve as major fuels for ATP production in myocardial cells. Acetyl CoA from these fuel molecules enter citric acid cycle to generate NADH and FADH2, whose electrons are transferred in the respiratory chain to oxygen with accompanying synthesis of ATP by a multiprotein complex. Cytochrome c is obligatory for the shuttling of electrons in the respiratory chain from cytochrome reductase to cytochrome oxidase before the transfer to oxygen. Whereas electron transport in the respiratory chain with the coupled oxidative phosphorylation in mitochondria provides far more ATP than it is possible in the absence of oxygen (e.g. via breakdown of glucose to pyruvate during glycolysis), it comes with a cost. For example one electron reduction of O₂ that produces the superoxide anion can cause radical reactions and oxidative modifications in protein, nucleic acid and lipid constituents in cells. Superoxide formation e.g. at the level of cytochrome oxidase is ordinarily in small quantities but can increase under adverse conditions. Various defenses exist to deal with the oxidative damage to cellular constituents but oxidative damage is still found to increase with increasing age in various cell types, including the myocardial cells that are essentially postmitotic in human. Their replacement e.g. by the progeny of stem cells in epicardium and elsewhere is inadequate or undetected when they are lost in significant quantities. Myocardium's dependence on the respiratory chain in mitochondria (i.e. dependence on a source of damage to it) for normal functions represents a dilemma manifested in the form of unsolved medical problems. Myocardial cell losses following ischemia-reperfusion can occur through necrosis or apoptosis depending on the duration and degree of ischemia and can lead to fibrosis and myocardial remodelling with consequent deteriorations of heart's pumping and related morbidities frequent in older adults. I have found that whereas Hh/Smo signaling in myocardial cells is essential for normal functioning of myocardium and is involved also in the interactions between myocardial and other cells in heart, a selective inhibitor of Hh/Smo signaling can be administered to a tumor bearing patient systemically in a dosing that causes elimination of the tumor cells from the patient without causing intolerable adverse effects on the cardiac and other normal functions of patient. The adverse effects determined to be difficult to avoid consequences of such dosing (e.g. nausea-vomiting and temporary hallucinations that may be related to the physiological uses of Hh/Smo signaling in brain) are tolerable and can also be controlled (e.g. with an antiemetic). Specifically concerning cardiovascular functions, electrocardiography, blood pressure and heart rate measurements and blood and urine samplings for laboratory analyses of cardiac molecular markers and indicators of such functions can be used for monitoring the patient and for adjusting the dosing for a particular patient. Lowering of blood pressure is recommended for hypertensive patients prior to initiating the tumor treatment by taking into account the individual needs of a patient. Pumping of heart against increased systolic blood pressure places extra demand on the ATP and oxygen needs of heart. Enzyme activity measurements in blood plasma or serum with appropriate isoenzyme determinations [e.g. of aspartate aminotransferase (AST), creatine kinase (CK) and CK-MB] can provide information about myocardium with adequate sensitivity and specificity. The same blood samples and other patient samples can be used for simultaneous determinations of other organ and tissue functions and effects thereto. Analytes suitable for that purpose are generally known in medical practice. Examples of testing and their results for the above mentioned lung cancer patient are given below.

Table 1 shows laboratory investigation results with blood sampled at about the 48^{th} hour from the starting of the systemic infusion of cyclopamine Alanine aminotransferase (ALT) activity in blood serum is known to be a sensitive indicator of hepatocyte damage and increases after such damage. It was normal in the patient. Normal amylase activity is consistent with lack of damage in pancreas, another organ known to depend on Hh/Smo signaling for normal functions. The elevated lactate dehydrogenase (LD) activity would be consistent with the induction of apoptosis of tumor cells as this is an enzyme that is typically highly expressed in tumor cells and known to be released from apoptosing cells. Aspartate aminotransferase (AST) is an enzyme present in high quantities in liver and muscle cells and relatively much less in most tissues. Its activity in the patient's blood serum was close to the upper reference range. ALT in blood serum is increased usually more than AST in cases of hepatocellular damage. The ALT and AST activities, considered together with the LD activity would also be against a detectable muscle cell damage as both AST and LD activities are known to be high in muscle cells (both in skeletal muscle and heart muscle AST is higher than LD). The slight elevation of bilirubin in blood serum involving mostly the direct bilirubin can be ascribed to the amount of the ethanol carrier administered. Normalcy of concentration in blood serum and red blood cell indices were consistent with lack of erythrocyte lysis or other damage and microscopic examination of blood smear also showed normal results. The elevated uric acid concentration in blood serum accorded with causation of efficient apoptotic removal of tumor cells from patient by the treatment. Its occurrence despite an adequate hydration of the patient would reflect the efficiency of induction of apoptosis of the tumor cells. Such efficiency can be complemented best by treatment with urate oxidase (or with another engineered enzyme for breakdown of uric acid to a water soluble product) as administration of such is known to be particularly effective and rapid in bringing down elevated uric acid concentrations in body fluids to obviate other measures. The laboratory investigation results of patient agreed with the clinical findings of patient that normal organ functions, including those known to be depended on Hh/Smo signalling, can be preserved while removing the tumor cells from patient with efficiency.

Hemangiomas that develop in adults are distinguished from the hemangiomas or malformations detected at birth with their age of occurrence and other features. They increase in frequency with increasing age. Often bright red in color, these are ordinarily small (about 1-2 mm diameter) tumors where tumor cells with features of vascular endothelial cells form numerous packed capillaries. As implied by their bright red color and revealed by histopathological and other investigations, the tumor cells in these tumors are continuously exposed to relatively high concentrations of oxygen brought by erythrocytes of arterial blood with which they are in close contact. As such these tumor cells may have effective defences against oxidative damage. An unusually large nodular hemangioma with history of rapid growth and size of about 6×7 mm was topically administered with a cream containing 18 mM cyclopamine at frequencies of about every 6 hours until a cumulation of about 25 micromole per cm² skin surface area. Such administration that had been determined to cause visible shrinkage of various other tumors was found to cause no detectable change of tumor size. Causation of decrease of the red color of the tumor on its superficial region was observed. The vascular nature of this tumor might have caused rapid diffusion away of the cyclopamine molecules reaching to the environs of tumor cells. Decrease of the red color of the tumor on its superficial region to a level like the rest of skin unlike the deeper portion remaining bright red could result from decrease of the blood circulation through the superficial region of the tumor that was exposed to relatively higher concentrations of cyclopamine. The topical applications were discontinued and the tumor was observed for several weeks without treatment. No decrease in the size of the tumor was found, indicating persistence of the tumor cells. At the end of this non-treated follow up, a solution of 18 mM cyclopamine in 98% ethanol, 2% phosphate buffered saline pH 7.4 was injected directly into the tumor with a needle of 0.4 mm external diameter while slowly withdrawing the needle to deliver about 50 μl at a time in frequencies of about every 8-12 hours for three days with monitoring. This means of manual injection is unlikely to provide a controlled rate slow delivery to the tumor and it along with the capillary nature of tumor would cause exposures of some erythrocytes and other cells to relatively high concentrations of the carrier ethanol exiting the needle to result in necrotic cell death. Darkening of areas of the tumor was also observed during this period in accord with such effect. The apparently necrosed part of tumor and epidermis above it was taken away to expose a thinned tumor behind. A cream containing 18 mM cyclopamine was applied onto the thinned tumor at frequencies of about every 6 hours in volumes of about 15 μl at each time. The tumor was found to become invisible with this treatment after about two days and the treatment was discontinued after two further days. The normal skin around the tumor that was exposed to the cyclopamine dose that caused the tumor to become undetectable did not show a sign of an adverse effect. Continued disappearance of the tumor and normal skin at the former site of the tumor was verified at follow up more than 3 years afterwards.

The results with a tumor of the endothelial cells of blood vessels are instructive also in view of the continued survival of tumor cells following causation of decrease of blood circulation of tumor. Whereas a drug treatment that provides disappearance of tumors from patients without recurrence constitutes a solution for a pressing problem, Hh/Smo signaling is needed also for continued maintenance of blood vessels and for angiogenesis in normal tissues. Aging is known to be associated with decreased circulatory efficiency and in this respect a nearly linear increase is detected in blood pressure with increasing age in human. Deterioration of circulation in patients with type II diabetes mellitus, a condition with various molecular and tissue structure findings consistent with accelerated aging, is also known. Blood circulation and blood pressure are affected by multiple metabolic variables as revealed conspicuously in investigations of type II diabetic patients (e.g. Taş S et al, Lancet 1994; 343:1194-1195) and the direct effects of Hh/Smo signaling on expressions of enzymes that control energy metabolism and expressions of secreted proteins that affect blood vessels (e.g. angiopoietin-1) require assessments of a dosing with a selective inhibitor of Hh/Smo signaling that provides elimination of tumor cells from patients also specifically concerning consequences on circulation through normal tissues. I have investigated this question by microscopic examinations and by functional tests. The results show that whereas a window of dosing exists within which tumor cells can be eliminated from tumor bearing patients, it is not wide and blood circulation through normal tissues is negatively affected by doses that exceed.

The selective inhibitor of Hh/Smo signaling used in the above exemplifications of the tumor treatment is not a molecule that causes damage to DNA when incubated with DNA. Investigations with tumor cells lacking normal p53 function have also shown that a dose of cyclopamine sufficing for induction of apoptosis of tumor cells in a patient acts without causation of damage to the genetic material. Causation of extensive damage of genetic material beyond repair can trigger apoptosis of tumor cells and it constitutes basis of many drug treatments of cancer that have long been practiced in medicine with varying success rates depending on the histopathological class and stage of tumor. Combining such drug treatments with an inhibitor of Hh/Smo signaling have been advocated in reviews of clinical trial results reported with various drug molecules that cause selective inhibition of Hh/Smo signaling. Administering an agent that causes damage to the genetic material is not recommended for a patient to be treated according to the treatment described here. Such agents can damage also the genetic material of normal cells of patient with irreversible negative effects particularly when unrepaired or misrepaired in stem cells. In addition, a tumor cell that may not undergo apoptosis upon exposure to such an agent for any one of several reasons (e.g. due to an acquired p53 pathway mutation) can be caused to undergo increased numbers of new mutations because of that exposure and the caused mutations can be in a gene encoding for a product involved in Hh/Smo signaling. For example a mutation in Smo at a site that affects binding to Smo of a drug molecule that acts by binding to that site may preclude use of that particular molecule in treatment of an affected patient. The determination in this invention that dosing of tumor bearing patients with a medicament comprised of a selective inhibitor of Hh/Smo signaling can be used to eliminate tumor cells from them independent of the histopathological class and stage of tumor, and without causation of damage in the genetic material in normal cells of patient, represents a solution to the problems of treatment of tumor bearing patients.

Causation of damage to the genetic material by various means such as by direct binding to DNA by a drug molecule or by interfering with normal functioning of an enzyme (e.g. topoisomerase) or other protein to cause such damage that in turn can induce apoptosis of an affected cell, which are drug treatment approaches that are widely used in treatments of tumor bearing patients today, must be distinguished from the safe breakdown of genetic material in the final execution stages of apoptosis. Unlike the numerous different molecular signals and context depended actions of them in regulation of survival of cells, the execution phase of apoptosis is similar or identical in different cell types whose apoptosis might have been induced by very different signals. An endonuclease that acts like DNAse I introduces cuts to DNA in chromatin in large numbers (e.g. Didenko V V et al, Journal of Cell Biology 1996; 135:1369-1376) in a process of breakdown of DNA all the way to uric acid. As a cell with such large numbers of cuts to DNA has no way of survival and other events during the execution phase of apoptosis also preclude cell survival and prepare an apoptosing cell to phagocytosis by macrophages and other cells, it is a safe process. The results of my investigations that I described above call for use of a drug molecule that does not cause damage to the genetic material in treatment of tumor bearing human by the presently described treatment. Causation of significant measurable damage to the genetic material by a molecule can be determined by assays well known in the field, including those that detect unrepaired DNA damage as described in the publications I have referred and by in vitro reporter assays of mutagenic effect, to determine whether a molecule that selectively inhibits Hh/Smo signaling avoids causation of such damage.

Patients having a tumor wherein Hh/Smo signaling is utilized for inhibition of differentiation and for inhibition of apoptosis of tumor cells can be treated effectively by the instant tumor treatment. Various methods are known for determination of Hh/Smo signaling in a tumor. Tumor biopsies are commonly used to confirm a tumor diagnosis in a patient and to determine its histopathological class. Part of such a biopsy can be used to determine expressions of Hh proteins (Shh, Ihh, Dhh) and proteins of direct Hh target genes (e.g. Patched1, Gli1, Gli2, Gli3 and others for which a suitable primary antibody is available for immunohistochemical detection). Another part can be frozen for analyses by in situ hybridization and quantitative determinations of specific RNA molecules encoding for Hh and for products of Hh target genes by PCR-based methods. Microscopic examinations of tumor tissue sections for determining the particular cells expressing a protein or RNA (of Hh, Smo and downstream proteins participating in Hh/Smo signaling, Hh target genes) can be particularly informative with regard to the particular means of activation of Hh/Smo signaling in a given tumor. Culturing of part of the freshly obtained tumor biopsy can also be done for tests of effects of a desired molecule. Loss-of-function mutations of Patched1, gain-of-function mutations of Smo and other mutations by which Hh/Smo signaling can be activated in tumor cells are known. Making high quantities of a Hh protein and responding to secreted Hh in autocrine-paracrine manner have also been found in some tumors. Secreted Hh proteins can act also for interactions between the neoplastic cells and various stromal cells in a tumor. Release of Hh proteins besides other signaling proteins by stromal cells and responses of neoplastic cells to them are also known. Hh proteins secreted by neoplastic cells can act also on stromal cells. Ligand independent activation of Hh/Smo signaling in neoplastic cells is commonly found in metastatic and late stage tumors and serves for inhibition of apoptosis of neoplastic cells in a foreign tissue environment. Metastatic tumors are formed by tumor cells that are surviving in a foreign tissue after leaving their tissue of origin.

The dosing of a tumor bearing patient according to the instant tumor treatment is directed to the elimination of tumor cells from the patient by induction of apoptosis of them as described. Tumor cells undergoing apoptosis can be determined by various methods. Microscopic examination of hematoxylin & eosin (H&E) stained sections of biopsied tissue for cells showing morphological characteristics of apoptosing cells can provide quantitative results with acceptable accuracy and precision (e.g. van de Schepop H A M et al, Journal of Clinical Pathology: Molecular Pathology 1996; 49:M214-M217). Immunohistochemical and other methods of in situ detection of the cells showing molecular markers of apoptosis are also known and can also be used. Tumor cells can be obtained from a patient by conventional biopsying, aspiration with ultrasonic guidance of a catheter or by other known means depending on a tumor site. In vivo imaging methods to visualize apoptosing cells are known and have the advantage of simultaneous visualization and measurement of tumor size. For example, in vivo imaging results using variously labeled annexin V have been described to show significant positive correlation with the results of histopathological determination of apoptosing cells and uses of other molecular markers and additional methods of in vivo imaging of apoptosing cells are also known (e.g. D'Arceuil H et al, Stroke 2000; 32:2692-2700; Blankenberg F et al, Journal of Nuclear Medicine 2001; 42:309-316). Blood sampling from a vein to measure concentration of suitable molecular markers released to the extracellular fluid and thereby to the blood plasma from apoptosing cells can be informative when used together with above mentioned methods for monitoring of induction of apoptosis of tumor cells in a patient.

Examinations of tumors in patients as above show frequencies of apoptosing tumor cells that do not exceed a few percent prior to treatment. Untreated tumors typically show continuous growth and not any efficiency of causation of apoptosis of tumor cells can provide a therapeutic result of tumor disappearance without recurrence. Growth status of a tumor depends on factors such as the frequency and rate of proliferation of tumor cells and frequency of tumor cell death. A tumor treatment, whether using a drug molecule that inhibits Hh/Smo signaling or not, may provide a slowing of tumor growth by causing decrease of proliferation of tumor cells and/or tumor cell death at a rate below the rate of input of new tumor cells. Disease stabilization by a treatment refers to a status in which the tumor does not show significant change of size during a period of observation. Tumor shrinkage occurs when a treatment causes tumor cell death with a rate surpassing the input of new tumor cells. Tumor disappearance refers to a result where a tumor becomes invisible by the employed method, commonly a radiographic imaging. Tumor disappearance without recurrence refers to elimination of the tumor cells from patient so that the tumor becomes undetectable and recurrence of the tumor does not occur in follow up for an appropriate duration. Recurrence of a tumor can be determined as such by histopathological examinations of a specimen from the detected tumor and if needed by comparisons with the original tumor by molecular genetic methods. Causation of new tumors by a treatment refers to a treatment that causes increased probability of occurrences of new tumors not originating from the first in the treated patients as determined by molecular genetic analyses of the normal cells of patients and by statistical analyses of clinical monitoring results.

In the investigations with tumor bearing patients as described above, not only the administered amount of the selective inhibitor of Hh/Smo signaling has been found to be critical with regard to the effects on tumor cells and normal cells but the duration during which the amount is administered has also been determined to affect causation of tumor disappearance without recurrence. Specifically, a dosing schedule where the selective inhibitor of Hh/Smo signaling is administered at a rate that is sufficiently high to cause efficient apoptotic removal of the tumor cells from patient with measures to avoid damage from it (e.g. due to the uric acid produced in significant quantities due to the efficiency of the apoptotic removal of tumor cells from a large tumor) has been determined to allow elimination of the tumor cells from patient. Dosing where daily administered amount of the selective inhibitor of Hh/Smo signaling is relatively decreased and the duration of administration is increased was determined not to be beneficial contrary to the expectations e.g. on safety grounds. The normal organ functions revealed by the clinical laboratory examination results as exemplified in Table 1 and by the described monitoring and other examinations following an effective dosing show that whereas Hh/Smo signaling is essential for normal functions, a selective inhibitor of Hh/Smo signaling can be used as described and it provides a solution to the problems of treatment of patients having a tumor that is not possible or desirable to remove by surgery or by other known treatment. Systemic administration of a medicament comprised of a selective inhibitor of Hh/Smo signaling for elimination of tumor cells by induction of apoptosis of them as described requires optimization of the dosing for a particular patient in consideration of his/her pre-treatment medical status. The mg/kg/day amount and rate of systemic infusion of a selective inhibitor of Hh/Smo signaling optimal for a given patient depends on his/her tumor load and pre-treatment status of organ functions. The above described specific findings and examples with patients provide guidance for optimal dosing of a given patient.

Liver and renal functions are generally involved in metabolism and excretions of drug molecules and it is known that other functions of a patient may also be needed to take into account in optimization of a dose of a medicament aiming to cause in him or her a previously known particular therapeutic effect. In case of a terminally ill cancer patient like in the lung cancer patient in the above example, a staged approach to improve first the general clinical condition of the patient and then to cause tumor disappearance can be followed.

Besides the assessments and monitoring of a patient as above, measurements of Hh/Smo signaling activity in suitable cells from patient (e.g. in skin cells, blood cells or others, e.g. by testing for expressions of one or more of Hh target genes such as patched1, gli1, gli2, gli3) can also be used in monitoring effects of a dosing in a patient.

A drug molecule suitable for use in the instant tumor treatment is a molecule that binds to a component of Hh/Smo signaling to provide selective inhibition of Hh/Smo signaling in an administered patient who has a tumor wherein Hh/Smo signaling is utilized for inhibition of apoptosis of tumor cells. Hh/Smo signaling is the intended target of the drug molecule and a selective inhibitor of Hh/Smo signaling is a molecule that does not have a primary effect on a non-intended molecular event in the patient to an extend to preclude inhibition of Hh/Smo signaling in the tumor of patient for induction of apoptosis of tumor cells for their elimination. Various screening assays are known for identifying molecules that inhibits Hh/Smo signaling in cells (e.g. Sasaki H et al, Development 1997; 124:1313-1322; Jiang K et al, Methods Mol Biol 2015; 1322:45-60). Methods to determine whether a molecule of interest identified in such a screen can provide selective inhibition of Hh/Smo signaling in experimental animals are also known (e.g. Ericson J et al, Cell 1996; 87:661-673; Incardona J P et al, Development 1998; 125:3553-3562; Stenkamp D L et al, Developmental Biology 2000; 220:238-252). Assays employing panels of human enzymes and transporters for predicting whether a molecule of interest would have a significant effect on an unintended physiological event in human are also known and various molecules identified through such a process have been described to provide selective inhibition of Hh/Smo signaling in tests in human (e.g. Goldman J et al, Clinical Cancer Research 2014; 21:1002-1009; Wagner A J et al, Clinical Cancer Research 2014; 21:1044-1051; Minami H et al, Cancer Science 2016; 107:1477-1483 and references therein). Because Smo is essential for transduction of the signaling initiated by Hh, many molecules have been made that bind to Smo to provide selective inhibition of Hh/Smo signaling in human. Sasaki N et al (Molecular and Cellular Neuroscience 2010; 45:335-344) describe methods for determining non-transcriptional effects of Smo-binding molecules. Molecules that bind to a component of Hh/Smo signaling other than Smo to provide selective inhibition of Hh/Smo signaling are also known and can be made (e.g. Ericson J et al, Cell 1996; 87:661-673). Pharmaceutically acceptable molecules that provide selective inhibition of Hh/Smo signaling can also be derived from cyclopamine for use in place of cyclopamine for practice of instant tumor treatment. A drug molecule suitable for use in place of cyclopamine for practice of instant tumor treatment is functionally restricted. It does not have a restriction of structural features as long as it provides selective inhibition of Hh/Smo signaling for use as described here. Automatable screening assays allow rapid determination of molecules that inhibit Hh/Smo signaling in cells without a priori restriction of structural features. A molecule found to inhibit Hh/Smo signaling with an IC₅₀ e.g. <10 μM in e.g. Gli-reporter assay (Sasaki H et al, ibid) can be checked in one or more of known whole animal models to determine whether or not it can provide selective inhibition of Hh/Smo signaling in an animal. Chicken embryo and zebrafish with defined mutations of genes encoding for proteins participating in Hh/Smo signaling (e.g. Incardona et al, ibid; Stenkamp et al, ibid) provide convenient models and allow determination of a molecule suitable for selective inhibition of Hh/Smo signaling without a restriction of molecule structure similar to the case with screening assays.

Binding of a molecule of interest to the Smoothened protein and its binding affinity can be determined by standard methods, such as by use of radiolabelled or otherwise labelled ligand and Scatchard analysis. By such methods Bijlsma M F et al (PLOS Biology 2006; e232) describe that cholesterol derived steroidal molecules that inhibit Hh/Smo signaling exist naturally in human and Vitamin D3 is such a molecule that inhibits Hh/Smo signaling by binding to the Smoothened protein with relatively high affinity to compete with cyclopamine, a steroidal molecule made by Veratrum plants. Vitamin D3 performs various physiological functions, including those related to calcium metabolism, by binding to a nuclear protein (Vitamin D receptor) that is different from Smoothened. Because concentrations of Vitamin D3 necessary for an inhibition of Hh/Smo signaling for causation of a therapeutic result as described in this invention are prohibitive in tumor bearing human due to e.g. the hypercalcemia that starts to risk life and can be lethal before approaching to a concentration required for removal of tumor cells from patient by induction of apoptosis of them, Vitamin D3 is not a molecule that is a selective inhibitor of Hh/Smo signaling. Synthesis of cyclopamine and related steroidal alkaloids in Veratrum plants evidently serves for defenses of plants against predators and cyclopamine exists in Veratrum plants along with many structurally related and unrelated alkaloids. Composite preparations of such plants had a long history of use in folk medicine and in early treatments of hypertension (e.g. Loofbourow D et al, New England Journal of Medicine 1950; 243:295-307; Long R S et al, Journal of the American Medical Association 1953; 152:1619-1622). Presence of numerous other molecules besides cyclopamine in whole plant parts and crude extracts and their diverse effects make them unsuited for a therapeutic use as described here.

Smo is essential for both transcriptional and non-transcriptional effects of the signaling initiated by Hh but molecules binding to different regions of its large intracellular C terminal can differentially affect the two so as to inhibit the Gli-mediated transcription without inhibiting one or more non-transcriptional effect (e.g. Sasaki N et al, Molecular and Cellular Neuroscience 2010; 45:335-344). Since Hh/Smo signaling supports maintenance of blood vessels and angiogenesis through both transcriptional and non-transcriptional effects and differentiated normal cells are in general more sensitive to ischemia than tumor cells, selective inhibitors of Hh/Smo signaling that inhibit the effects of Smo on the Gli-mediated transcription without inhibiting a non-transcriptional Smo effect that supports maintenance of blood vessels can be identified for use in the instant tumor treatment. Such a Smo inhibitor can be used more beneficially in the instant tumor treatment in place of cyclopamine. Convenient in vitro methods for determining effects of a selective inhibitor of Hh/Smo signaling on capillary formation by normal endothelial cells and whether it can provide a dissociation between the non-transcriptional and Gli-mediated effects of Smo are known (e.g. Kanda S et al, Journal of Biological Chemistry 2003; 278:8244-8249) and several animal models for quantitative determination of in vivo effects of an identified Smo inhibitor on blood vessel maintenance and angiogenesis are also known (e.g. corneal angiogenesis assay).

The aforementioned screening assays, animal models and known methods of testing with panels of human proteins allow also identifications of molecules that can be used for selectively activating Hh/Smo signaling in vivo in human. The process provides the suitable molecules again without a restriction of structural features. A molecule suitable for selectively activating Hh/Smo signaling in vivo in human can be one that binds to Smo to provide selective activation of Hh/Smo signaling but a pharmaceutically acceptable molecule that provides selective activation of Hh/Smo signaling by binding with another component of Hh/Smo signaling can also be used.

A medicament comprised of a selective inhibitor of Hh/Smo signaling (or one comprised of a selective activator of Hh/Smo signaling for a treatment pointed below that employs such an agonist) can be formulated for administration by a systemic route or for topical administration or for another means of preferential action at a tissue or organ site to which the medicament is administered, e.g. by injection. Formulations that allow controlled release of a drug molecule are known. Incorporation of a drug molecule to liposomes can provide improved delivery of a drug molecule to a site of intended action. Principles and methods of formulating pharmaceutical compositions for various routes of delivery are known in pharmacology and pharmaceutical sciences (e.g. the textbook, Remington's Pharmaceutical Sciences).

Elimination of tumor cells from patients by the described treatment with safety sparing the normal cells, including the normal stem cells, and without causation of damage to the genetic material, serves for effective treatments of certain pathological conditions that show increase of frequency with aging. For example, osteoporosis and its complications that are frequent in post-menopausal women are known to be beneficially treated by estrogen replacement therapy that has other benefits as well on several other tissues, organs and functions of aging women but applicability of such replacement is restricted largely due to the increased risk of breast cancer and other cancers with such replacement. The capability of effectively and safely eliminating tumor cells from patients by the present tumor treatment can be used for, and paves the way for, effective treatments of such pathological conditions experienced by aging women.

Longitudinal and cross sectional investigations show that frequencies of various pathological conditions affecting multiple organs increase with aging in human. Effective treatments are known for few of them. Whereas treatment efforts may aim each such disease individually as it is often the case, analyses of pathogenesis point to particular age associated alterations in postmitotic cells as well as in cells that can proliferate as root causes in several age associated diseases. Specifically, I found that age associated increase of unrepaired damage in DNA and the age associated decrease of efficiency of repair and failures of repair following damaging at increased levels are both related to constraints imposed by chromatin structure on repair. Unrepaired and misrepaired damage in surviving cells have pathological effects and increased apoptotic losses of normal cells upon excessive damage of genetic material also have pathological effects. Increased losses of postmitotic neurons in Alzheimer's disease and of myocardial cells in various age associated cardiac pathologies are examples of latter. Lymphocytes are long lived cells that proliferate infrequently under normal circumstances in human. I found that indicators of unrepaired damage in genetic material increase in these cells with increasing donor age in apparently healthy men and women and the age related alterations of chromatin structure that were found earlier in lymphocytes, hepatocytes and other cells in mice are detected also in human lymphocytes. The questions of how and why the genetic material shows accrual of damage with aging have been inadequately addressed. I pointed earlier that heterochromatinization occurs in chromatin structure of genes that are silenced during cellular differentiation and most of repeated sequences containing repeats of transposable elements are constitutively heterochromatinic and such chromatin structure negatively affects repair of particular types of damage to DNA. Failures of repair in the genomic regions containing active retrotransposon sequences have the additional risk of leading to loss of silencing to cause new transpositions and damaging of genetic material. Active retrotransposon sequences in human and molecular genetic methods for their deletion and inactivation are known. Both prevention and effective repair of damage to the genetic material are beneficial. I have found earlier that exogenous supply of suitable sulfhydryl molecules to lymphocytes can improve their functional performances and significantly more with lymphocytes from older than younger adults (Taş S et al, Mechanisms of Ageing and Development 1982; 19:73-84). Decrease of intracellular concentrations of glutathione (GSH; an endogenous sulfhydryl molecule) is found to cause increases in measurable oxidative damage and to preclude cell survival when decrease of GSH is excessive. Evidence is now found that Hh/Smo signaling can directly affect redox metabolism and it can be used for decrease of oxidative damage in cells in accord with the effects of varying concentrations of cyclopamine on normal stem cells and on their progeny at their natural tissue sites. Normal stem cells, supported by niche cells with relatively high concentrations of Hh, are in general found to have better protection from damage to the genetic material than their differentiated progeny.

These findings have practical impact in uses of normal stem cells for regeneration of tissues and for uses thereof in treatments of age associated pathologies such as those resulting from increased cell losses in the central nervous system and in heart. Increase of damage to the genetic material, in particular the oxidative damage, is found to be instrumental in the increased losses of postmitotic cells in older adults. Differentiated progeny of normal stem cells normally provide replacements for apoptosing normal cells in many tissues but functional and histopathological examinations show that proper replacement and tissue repair typically fail in aging brain and heart. Normal stem cells, potential resources for tissue repair, are also found not to escape the age associated increase of damage in the genetic material although they are in general better protected than their differentiated progeny. Stem cells prepared from various tissues of older adults are in this respect found to show usually poorer performance than those prepared from embryos, infants and younger adults in functional assays and attempts of using normal stem cells from younger subjects for therapeutic purposes face the problems of histoincompatibility. Normal stem cells in which active retrotransposon sequences have been deleted and inactivated by molecular genetic methods can be expanded as such stem cells to provide improved cells for therapeutic use.

TABLE 1
Clinical Laboratory Test Results Showing Preservation Of Normal
Cells and Normal Organ Functions Of Tumor Bearing Patient
Following Systemic Dosing With A Medicament Comprised Of A
Selective Inhibitor Of Hedgehog/Smoothened Signaling

| | | |
|---|---|---|
| | Result Of | |
| | Measurement | Reference |
| Analyte | In Patient | Range |

| | | | |
|---|---|---|---|
| Alanine aminotransferase | 35 | IU/L | 5-41 |
| Amylase | 30 | IU/L | <90 |
| Aspartate aminotransferase | 47 | IU/L | 6-38 |
| Lactate dehydrogenase | 1070 | IU/L | 240-480 |
| Uric acid | 13.2 | mg/dL | 3.4-7.0 |
| Total bilirubin | 2.33 | mg/dL | <1.1 |
| Direct bilirubin | 1.57 | mg/dL | <0.3 |
| K⁺ | 4.59 | mM | 3.5-5.5 |

| | | |
|---|---|---|
| Erythrocyte count | 4.43 × 10⁶/μL | 4.00-5.80 |

| | | | |
|---|---|---|---|
| Hemoglobin | 11.7 | g/dL | 12.0-17.5 |

| | | |
|---|---|---|
| White blood cell count | 11.5 × 10³/μL | 4.5-11.0 |

Blood samples of the lung cancer patient described in the exemplification were analysed following non-oral systemic dosing as described with a medicament comprised of cyclopamine (18 mM) in 98% ethanol, 2% phosphate buffered saline pH 7.4.

1-9. (canceled)

10. A method for treatment of a patient having a tumor, comprising
determining that the tumor in the patient is a tumor wherein Hedgehog/Smoothened signaling is utilized for inhibition of apoptosis of tumor cells, and
administering to the subject a medicament comprised of a pharmaceutically acceptable molecule that selectively inhibits Hedgehog/Smoothened signaling,
wherein said medicament is administered in a dosing that is sufficient to cause apoptosis of said tumor cells and elimination of the tumor cells from patient independent of the histopathological class and stage of tumor.

11. A method according to claim 10, wherein said administering is a systemic infusion or an injection to the tumor or a topical application.

12. A method according to claim 10, wherein the patient is determined to show disappearance of the tumor without recurrence and to show normal stem cells and functions that are dependent on Hedgehog/Smoothened signaling.

13-14. (canceled)

15. A method according to claim 10, wherein said molecule binds to the Smoothened protein to cause said inhibition.

16. A method according to claim 10, wherein said molecule binds to the Smoothened protein to inhibit Smoothened's activation of Gli-mediated transcription and does not inhibit a non-transcriptional effect of Smoothened that supports angiogenesis and blood vessel maintenance.

17. A method according to claim 10, wherein said molecule is cyclopamine.

18. A method according to claim 10, wherein said molecule is in a sterile aqueous or nonaqueous solution or in a microemulsion, or it is in a powder form for reconstitution to a solution prior to administration, or it is in a pharmaceutical form enabling controlled release, or incorporated to liposomes, or it is in a cream or ointment or gel or hydrogel or adsorbed onto a dermal patch.

19-30. (canceled)

31. A medicament for treatment of a human having a tumor wherein Hedgehog/Smoothened signaling is utilized for inhibition of apoptosis of tumor cells,
comprising a drug molecule that selectively inhibits the transcriptional effects of Hedgehog/Smoothened signaling and does not inhibit a Smoothened-mediated non-transcriptional effect that supports blood vessel maintenance and/or angiogenesis,
wherein the medicament is administered in a dosing that causes apoptosis of the tumor cells and tumor disappearance.

32. A medicament according to claim 28, wherein said administration of medicament is directly to tumor with aid of an endoscope in a sufficiently high amount and frequency that causes said apoptosis of tumor cells and disappearance of tumor.

33-51. (canceled)

52. A method for causation of tumor disappearance without recurrence in a tumor bearing human who has a tumor that has metastases or a location or invasions that make it unsuitable for excision from the patient by surgery to cause tumor disappearance without recurrence, comprising
determining that the tumor in the patient is a tumor wherein Hedgehog/Smoothened signaling is utilized for inhibition of apoptosis of tumor cells, and
administering to the patient a medicament comprised of a drug molecule that selectively inhibits Hedgehog/Smoothened signaling,
wherein the medicament has a pharmaceutical form and amount of the molecule adequate for administration in a dosing for causing apoptosis of the tumor cells and visual disappearance of tumor, and
is administered in a sufficiently high amount and rate and frequency determined to cause apoptosis of the tumor cells with efficiency characterized by causation of empty spaces devoid of tumor cells within the tumor or by causation of a decrease of size of the tumor by day 5 of the starting of administration, and
said dosing continues until the tumor becomes invisible and continues after said invisibility for an additional duration at the end of which the normal stem cells of patient exposed to the administered medicament start to decrease in number relative to that at the time of occurrence of said invisibility.

53. A method according to claim 52, wherein said administration causes invisibility of the tumor by day 12 of the starting of administration.

54. A method according to claim 52, wherein said administration of medicament is by systemic infusion.

55. A method according to claim 52, wherein said administration of medicament is by systemic infusion and the patient is monitored for cardiac functions during administration and measures are taken to lower the cardiac demand for oxygen during administration, and the uric acid concentration in the patient's body fluids is monitored for taking measures for its excretion from patient without precipitation in tissues.

56. A method according to claim 52, wherein said administration of medicament is topical or by intratumoral injection.

57. A method according to claim 52, wherein the normal stem cells are identified by their expression of the Epithelial Cell Adhesion Molecule (Ep-CAM).

58. A method according to claim 52, wherein the patient is periodically checked after completing the medicament administration and determined to show continued tumor disappearance at follow up more than three years after the treatment.

59-66. (canceled)

67. A method according to claim 52, wherein said molecule binds to the Smoothened protein to cause said inhibition.

68. (canceled)

69. A method according to claim 52, wherein said molecule is in a sterile aqueous or nonaqueous solution or in a microemulsion, or it is in a powder form for reconstitution to a solution prior to administration, or it is in a pharmaceutical form enabling controlled release, or incorporated to liposomes or it is in a cream or ointment or gel or hydrogel or adsorbed onto a dermal patch.

70. A method according to claim 10, wherein said administration of medicament is directly to tumor with aid of an endoscope in a sufficiently high amount and frequency that causes said apoptosis of tumor cells and disappearance of tumor.

71. A method according to claim 52, wherein said administration of medicament is directly to tumor with aid of an endoscope in a sufficiently high amount and frequency that causes said apoptosis of tumor cells and disappearance of tumor.
